(19) United States
(12) Patent Application Publication
(10) Pub. No.: US 2016/0175134 A1
(43) Pub. Date: Jun. 23, 2016

(54) Weight Acceptance Control Orthosis

(71) Applicant: YALE UNIVERSITY, New Haven, CT (US)

(72) Inventors: Kamran Shamaei Ghahfarokhi, New Haven, CT (US); Aaron Michael Dollar, New Haven, CT (US)

(21) Appl. No.: 14/976,687

(22) Filed: Dec. 21, 2015

Related U.S. Application Data
(60) Provisional application No. 62/094,456, filed on Dec. 19, 2014.

(51) Int. Cl. A61F 5/01

(52) U.S. Cl. CPC A61F 5/0102; A61F 5/0123; A61F 5/0127; A61F 5/013; A61F 2005/0188; A61F 2005/0137; A61F 2005/0146; A61F 2005/0162; A61F 2005/0179

(57) ABSTRACT

In certain aspects, the present invention is a purely mechanical stance control orthosis, or stated differently, a weight acceptance control orthosis, as it stabilizes the impaired knee joint during the first 40% of the gait cycles termed as the weight acceptance phase. The device employs a tri-stable fixture to lock/unlock a friction-based latch to provide two levels of stiffness. The device is locked during the weight acceptance phase to stabilize the impaired knee joint for an orthotic application or avoid collapse for a prosthetic application, and is unlocked during the rest to allow free rotation. The device includes an engagement fixture, a locking mechanism, a chassis, and a frame. The engagement fixture controls the locking mechanism to lock and unlock movement in the chassis and frame in response to a wearer's weight load and is capable of locking at any angle.

### CROSS-REFERENCE TO RELATED APPLICATIONS

This application claims priority to U.S. Provisional Patent Application No. 62/094,456 filed Dec. 19, 2014, the contents of which are incorporated by reference herein in their entirety.

### STATEMENT REGARDING FEDERALLY SPONSORED RESEARCH OR DEVELOPMENT

This invention was made with government support under grant No. W81XWH-11-2-0054 awarded by the United States Defense Medical Research Development Program. The Government has certain rights in the invention.

### BACKGROUND OF THE INVENTION

Millions of patients worldwide suffer from hip, knee, and ankle joint disorders, including quadriceps weakness, Patellofemoral Pain Syndrome, or from injuries, stroke, post-polio, multiples sclerosis, or SCI. An improvement in lower extremity assistive devices will benefit some or all of these patients. However, with few exceptions, orthotic options for this population are limited to technologies that cannot provide assistance necessary to replicate the function of an unaffected limb. Accordingly, there is great potential for the development of orthosis devices to drastically increase the quality of life of this population. Gait pathologies and musculoskeletal disorders are often stabilized using a leg orthosis, typically consisting of a crude hard piece of material formed to the wearer's leg. Recently, new orthotic technologies have been introduced that employ external actuators and powered systems to rigidly lock the knee during the stance phase and unlock it during the swing phase of the gait. External actuators and powered systems are generally bulky and often result in low gait speed, joint pain due to additional load, and/or an unnatural gait. Powered systems require a power source, and are prone to faulty operation if the power system dies or malfunctions. Furthermore, powered orthotic devices require sensors and electronics, which have not shown the level of reliability and endurance that is required for medical applications. Thus, there is a need in the art for a small-size purely mechanical orthotic device that does not require an external power source, sensors and electronics.

### SUMMARY OF THE INVENTION

The invention is a purely mechanical passive orthotic joint that utilizes a friction-based latch manipulated by a tri-state mechanism. The orthotic joint allows for impedance modulation in that the knee joint is compliantly supported during the weight acceptance phase and dampens downward forces for the user, and the knee joint is allowed free rotation during the swing phase. The present invention solves issues associated with powered and electronic orthoses, has a small size and light weight, it can be used bilaterally, is passive and mechanical and can be used in any environment of the daily life, and has high endurance and reliability. Moreover, the device according to aspects of the invention can be used for prosthetic applications as a knee-ankle-foot or knee-ankle prosthesis.

In one aspect, the present invention relates to a friction-based impedance module, comprising: an engagement fixture, a locking mechanism, a chassis, and a lower frame, wherein the chassis is connected to the lower frame by a pivoting joint, wherein the module is configured to lock the pivoting joint responsive to an increased torque load, and wherein the module is configured to unlock responsive to a decreased torque load.

In one embodiment, the locking mechanism is at least partially housed within the engagement fixture. In one embodiment, the locking mechanism comprises a plunger, a friction trail, and a friction lever with a trail slot. In one embodiment, the friction trail passes through the trail slot of the friction lever. In one embodiment, the alignment of the trail slot with respect to the friction trail is controlled by the engagement fixture. In one embodiment, the trail slot contacts the friction trail to lock the pivoting joint. In one embodiment, the friction trail has a protrusion or void at an angle that prompts the engagement fixture into an engaged configuration. In one embodiment, the friction lever is released from the engaged configuration by a magnet, a spring, a solenoid, gravity, a motor, or movement of a user's limbs. In one embodiment, the friction-based impedance module further comprises a mechanism for delaying the unlocking of the friction-based impedance module in response to a decreased torque load.

In one embodiment, the locking mechanism is selected from the group consisting of: a ratchet and pawl mechanism, a roller clutch mechanism, a hydraulic mechanism and a wrap spring clutch mechanism. In one embodiment, the friction-based impedance module further comprises at least one of a safety lock, configuration lock, dials, adjustment knobs, range of motion limits, dampers, and lubricating system. In one embodiment, the spring is replaced by a rubber band or other elastic material.

In another aspect, the present invention relates to a hip-knee-ankle-foot orthosis (HKAFO), hip-knee-orthosis (HKO), knee-ankle-foot orthosis (KAFO), ankle foot orthosis (AFO), hip orthosis (HO), shoulder orthosis (SO), elbow orthosis (EO), wrist orthosis (WO), or knee orthosis (KO) comprising the friction-based impedance module of the present invention.

In another aspect, the present invention relates to a serpentine spring device comprising a two-dimensional plate having at least one S-shaped region. In one embodiment, the serpentine spring is composed of at least one material selected from the group consisting of: carbon fiber, a metal, and a composite material. In one embodiment, the serpentine spring further comprises at least one additional stacked plate to modulate the flexibility of the device.

In another aspect, the present invention relates to an orthotic device, comprising: an impedance module including a chassis, a lower frame, and a walking member; wherein the chassis is coupled to the lower frame by a pivoting joint; and wherein the walking member is coupled to the lower frame by a serpentine spring.

In one embodiment, the spring comprises at least one of a carbon fiber, metal, or composite material. In one embodiment, the impedance module further comprises an engagement fixture and a locking mechanism, such that the module is configured to lock the pivoting joint responsive to an increased torque load, and wherein the module is configured to unlock responsive to a decreased torque load.

In one embodiment, the locking mechanism is at least partially housed within the engagement fixture. In one embodiment, the locking mechanism comprises a plunger, a friction trail, and a friction lever with a trail slot. In one embodiment, the friction trail passes through the trail slot of the friction lever. In one embodiment, the alignment of the trail slot with respect to the friction trail is controlled by the engagement fixture. In one embodiment, the trail slot contacts the friction trail to lock the pivoting joint. In one embodiment, the friction trail is released by a magnet, a spring, a solenoid, gravity, a motor, or movement of a user's limbs. In one embodiment, the locking mechanism is selected from the group consisting of: a ratchet and pawl mechanism, a roller clutch mechanism, a hydraulic mechanism and a wrap spring clutch mechanism.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments, which are presently preferred. It should be understood, however, that the invention is not limited to the precise arrangements and instrumentalities of the embodiments shown in the drawings.

FIG. 1 depicts an exploded view of a friction-based impedance module incorporating a locking mechanism according to an exemplary embodiment of the invention.

FIG. 2 depicts a schematic of a plunger for use with the exemplary friction-based impedance module.

FIG. 3 depicts a schematic of a chassis for use with the exemplary friction-based impedance module.

FIG. 4 depicts a knee-ankle-foot orthosis equipped with the exemplary friction-based impedance module.

FIG. 5A through FIG. 5D depict the operation of an exemplary friction-based impedance module through different phases of the gait cycle.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical orthosis devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

The present invention includes a mechanical friction-based impedance modulation device (weight acceptance control orthosis) for orthotic applications. The device can function in parallel with any upper or lower extremity joints, such as the elbow or knee joint for example, to replace or supplement the function of an impaired joint. The device locks in response to a torque, such as a torque caused by rotation or a user's weight load on a joint, and subsequently unlocks upon removal of the torque to restore full range of motion to the joint. The device is capable of locking at any angle in response to a torque.

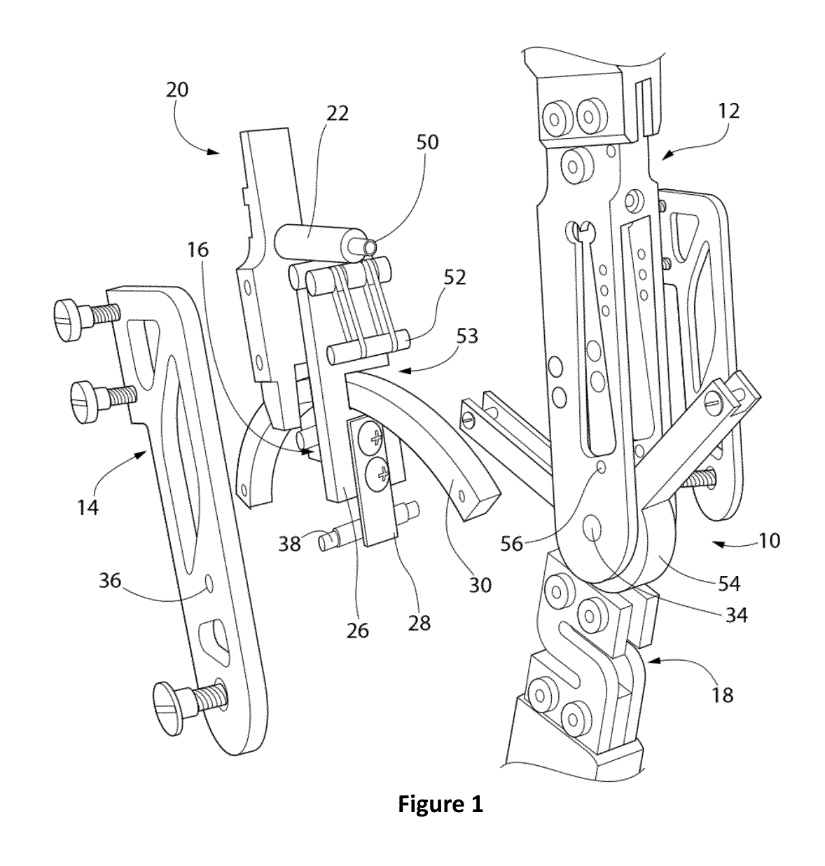

Referring now to FIG. 1, an exploded view of an exemplary friction-based impedance module 10 is shown. The friction-based impedance module 10 includes an engagement fixture 14, a locking mechanism 20, and a chassis 12. As shown, the engagement fixture 14 includes side frame components 1 and 3, and a rear frame component 2. The distal portion of friction-based impedance module 10 includes a stopper rod 56, and a lower frame component 54 connected to a serpentine spring 18. Locking mechanism 20 includes a plunger 22, a plunger spring 50, a posterior rod 40, an anterior rod 42, a rubber band anchor rod 52, at least one rubber band 24, a magnet 16, a friction lever 26, a leaf spring 28, a friction trail 30, and a pin 38. Friction lever 26 and rear frame component 2 each include a trail slot 53.

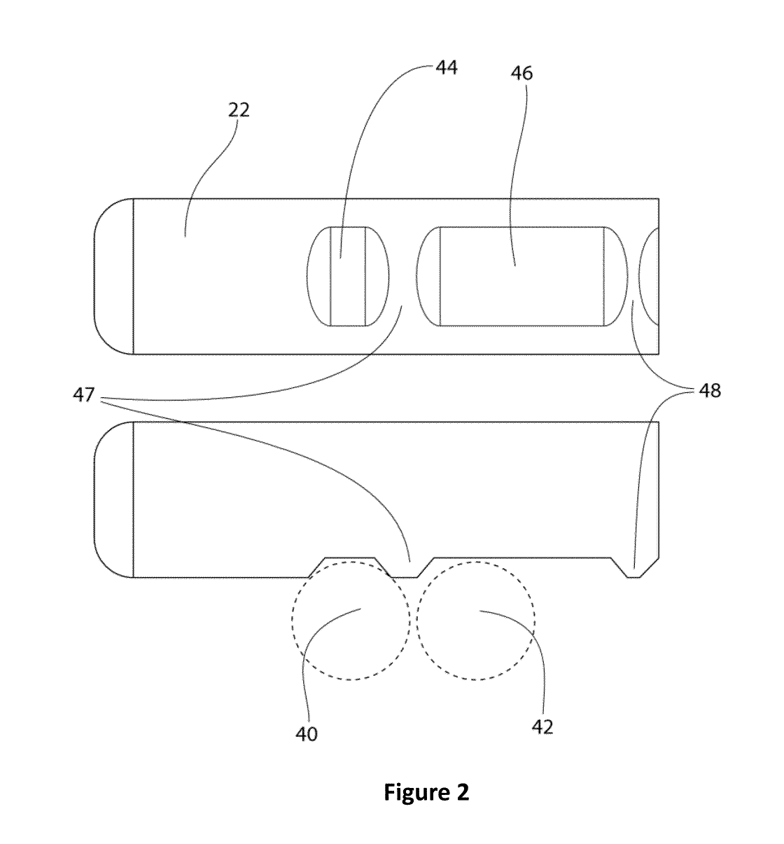

Referring now to FIG. 2, a schematic of plunger 22 is shown. Plunger 22 includes a short notch 44 and a long notch 46, thereby forming a posterior ridge 47 between short and long notches 44 and 46. At the opposing end of long notch 46 is an anterior ridge 48.

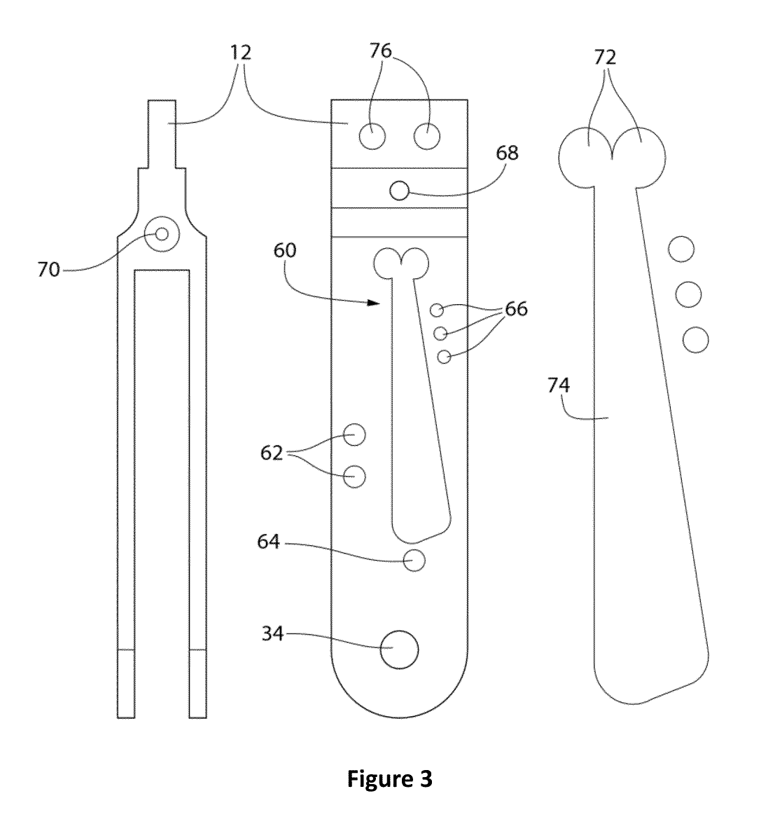

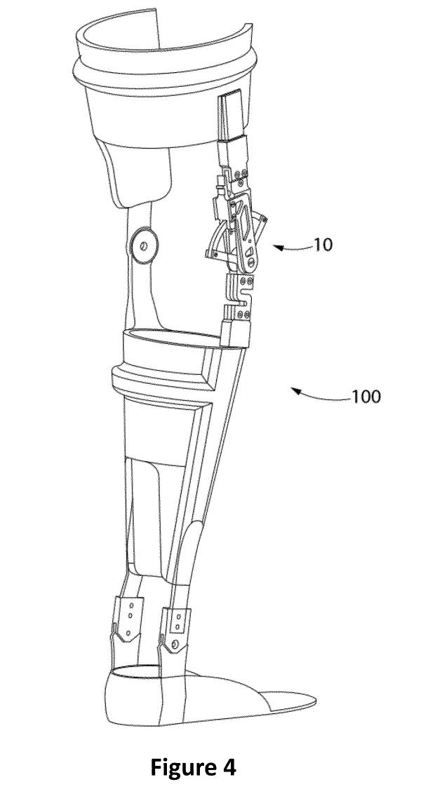

Referring now to FIG. 3, a schematic of chassis 12 is shown. Chassis 12 includes one or more orthotic attachment holes 76, a spring attachment hole 68, a tunnel 70, a slot 60, one or more adjustment holes 66, one or more magnet slots 62, a stopper slot 64, and a hole 34 to form a joint. Attachment points 76 are located on the proximal end of chassis 12 for securing chassis 12 to, for example, an orthotic device 100 as shown in FIG. 4. Tunnel 70 runs through chassis 12 and is open on both the anterior and posterior ends. Slot 60 includes plunger rod guides 72 and friction lever guide 74.

As illustrated, side frames 1 and 3, and rear frame 2 are connected to each other via pin 38 engaging holes 36, such that engagement fixture 14 moves as a single unit. In such an embodiment, at least one rubber band 24 loops around anterior rod 42 and rubber band anchor 52. The position of rubber band anchor 52 may be adjustable by inserting it in any one of adjustment points 66 of chassis 12. Accordingly, adjusting the position of rubber band anchor 52 also adjusts the force that the at least one rubber band 24 exerts upon anterior rod 42.

Leaf spring 28 is attached to the distal end of friction lever 26, such that leaf spring 28 and friction lever 26 move as a single unit. Accordingly, pin 38 resists the range of movement by leaf spring 28 when pin 38 is engaged with holes 36. Further, friction lever 26 is positioned to engage with chassis 12 such that it fits into friction lever guide 74 of slot 60. Thus, the proximal edge of friction lever 26 rests against posterior rod 40 and anterior rod 42. The selected magnet slot 62 holds or otherwise engages magnet 16. Accordingly, the position of magnet 16 is also adjustable by inserting it in any of magnet slots 62. Once in position, magnet 16 magnetically attracts friction lever 26.

As shown, friction trail 30 is attached to the extended arms of lower frame 54, thereby allowing friction trail 30 and frame 54 to move as a single unit. In this configuration, friction trail 30 passes through trail slot 53 of friction lever 26. Accordingly, engagement fixture 14, chassis 12, and lower frame 54 may be joined together at joint 34. As shown, engagement fixture 14, chassis 12, and lower frame 54 pivot independently about joint 34. For example, lower frame 54 may pivot in flexion to move friction trail 30 in an anterior direction, or lower frame 54 may pivot in extension to move friction trail 30 in a posterior direction. As such, stopper rod 56 limits the range of pivot by lower frame 54.

Friction trail 30 provides the friction for locking exemplary friction-based impedance module 10 depicted in FIG. 1, as described elsewhere herein. It should be appreciated that the present invention may be locked with any suitable mechanism that provides a friction. Non-limiting examples of suitable mechanisms include: a ratchet and pawl; a roller clutch, a wrap spring clutch, hydraulic mechanisms, and the like.

As shown in FIG. 1, serpentine spring 18 is attached to the distal end of lower frame 54. In one embodiment, the distal end of serpentine spring 18 is attached to, for example, an orthotic or prosthetic device, such that serpentine spring 18 dampens the force exerted on a wearer while using the orthotic or prosthetic device. Accordingly, serpentine spring 18 enables torsion in, for example, an orthotic device as shown in FIG. 4. Serpentine spring 18 can be made of any suitable material. For example, non-limiting materials include carbon fiber, metals, composites, and the like. In certain embodiments, the serpentine spring is S-shaped, and includes two two-dimensional S-shaped plates sandwiching the lower frame 54. In other embodiments, the serpentine spring may comprise a plate having a plurality of S-shaped regions in linear succession to increase the length of the serpentine spring. In other embodiments, additional plates may be any desired shape, meaning the additional plates may or may not be S-shaped. The spring constant can be modified by either adding more plates, or customizing the constant of each of the base plates. In certain embodiments, the torque of the serpentine spring can be modified by adding one or more spring laminae. For example, spring laminae may be stacked onto the serpentine spring to adjust the torque based on spring laminae orientation and constraint. The spring laminae may be any desired length and shape, depending on the position of laminae when stacked. Accordingly, the serpentine spring device may be modified with any number of additional plates and/or laminae, to effectively modulate the flexibility of the spring, to facilitate adjustment of torque or differentiation of torque for clockwise verses counterclockwise rotations. As shown in FIG. 1 and FIG. 4, the exemplary embodiment provides for easy switch-out, replacement, customization and installation of serpentine springs without the need for disassembling the engagement fixture 14.

In one embodiment, plunger 22 is housed within tunnel 70 of chassis 12, and the posterior end of plunger 22 rests against rear frame component 2. Further, a spring wraps around the proximal edge of rear frame 2 and is anchored to chassis 12 by spring attachment 68. Accordingly, the spring anchored by spring attachment 68 maintains the contact between rear frame 2 and the posterior end of plunger 22. In one embodiment, the anterior end of tunnel 70 is plugged by a threaded screw. In one embodiment, plunger spring 50 is compressed within tunnel 70 between plunger 22 and a threaded screw.

Posterior rod 40 and anterior rod 42 fit into plunger rod guides 72 of slot 60. In one embodiment, plunger rod guides 72 are dimensioned such that they permit posterior rod 40 and anterior rod 42 to move in only the proximal and distal directions. In this configuration, posterior rod 40 holds the position of plunger 22. For instance, posterior rod 40 may rest in short notch 44 against posterior ridge 47 to maintain plunger 22 in an anterior position. In another instance, posterior rod 40 may rest in long notch 46 against anterior ridge 48 to maintain plunger 22 in a posterior position. Alternatively, anterior rod 42 holds the position of plunger 22. For instance, anterior rod 42 may rest in long notch 46 against anterior ridge 48 to maintain plunger 22 in a medial position.

Friction-based impedance module 10 is amenable for use in any application wherein the function of a joint, such as an impaired joint, is in need of having its function replaced or supplemented. In particular, friction-based impedance module 10 can be used in an orthosis to replace or supplement the function of a joint. Friction-based impedance module 10 can also be used to replicate the function of a joint in a prosthetic. Suitable joints include those of the lower extremities and the higher extremities, such as the hip, the knee, the ankle, the wrist, the elbow, the shoulder, and the like. Accordingly, any embodiment of friction-based impedance module 10, any embodiment of serpentine spring 18, either separately or in combination, may be incorporated into any type of orthosis device. Example orthoses include hip-knee-ankle-foot orthoses (HKAFO), hip-knee-orthoses (HKO), hip-knee-ankle orthoses (HKAO), knee-ankle-foot orthoses (KAFO), knee-ankle orthoses (KAO), ankle-foot orthoses (AFO), knee orthoses (KO), hip orthoses (HO), shoulder orthoses (SO), elbow orthoses (EO), wrist orthoses (WO), and the like.

It should be understood that the devices of the present invention may comprise any additional elements that enhance the function or safety of the devices. For example, the additional elements may improve device performance, customization, and ease of use. Non-limiting examples of such elements include safety locks, configuration locks, dials, adjustment knobs, range of motion limits, dampers, lubricating systems, and the like.

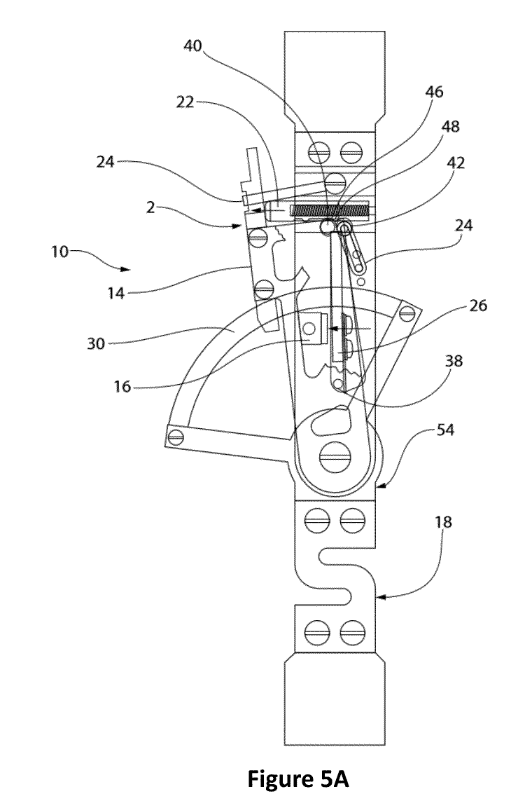

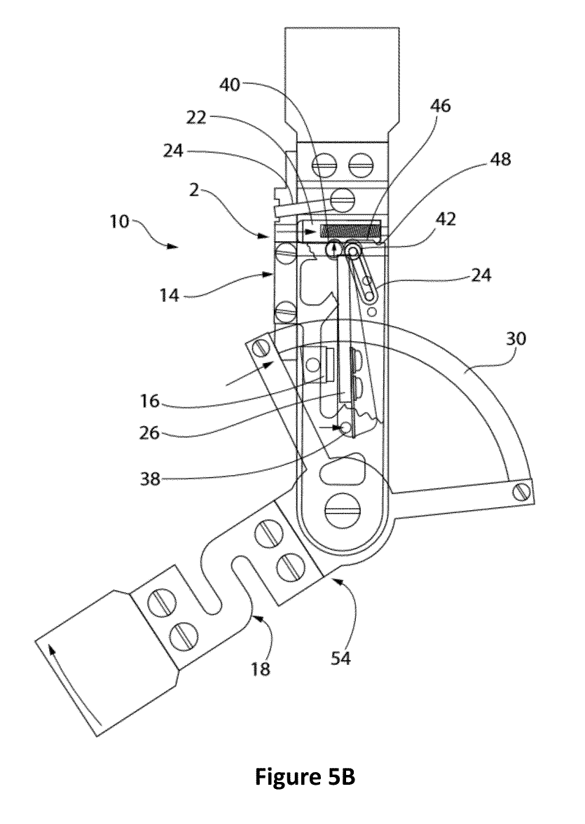

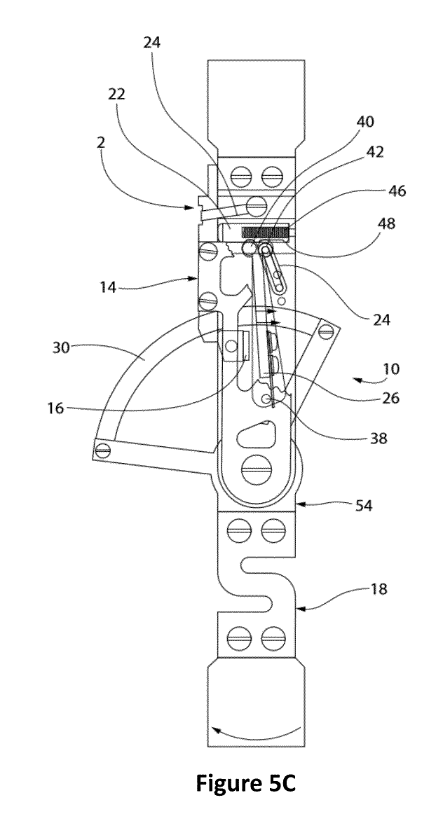

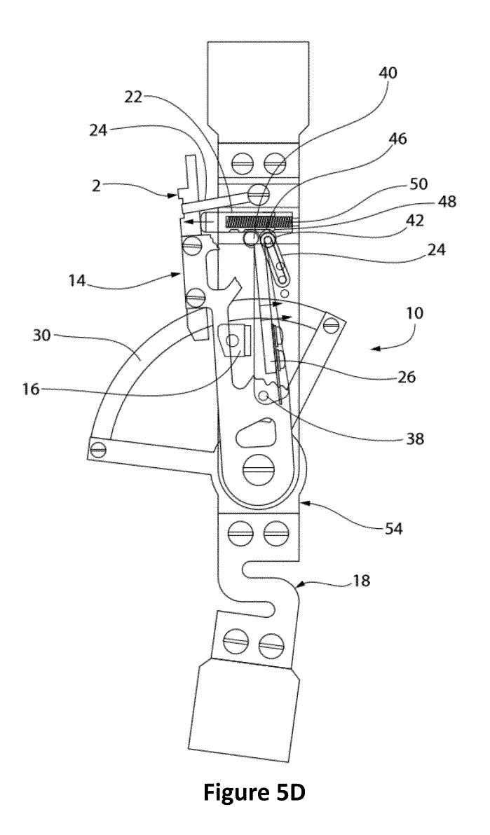

FIG. 5A through FIG. 5D track the operation of an exemplary friction-based impedance module 10 through different phases of the gait cycle (with details of certain elements described below shown in FIG. 1 through FIG. 3). In a first state that starts exactly after the weight acceptance phase ends around ˜40% through the gait cycle and ends in the swing phase when the knee maximally flexes, the friction-based impedance module 10 is unlocked. At the start of this phase and with reference to FIG. 5A, the proximal edge of friction lever 26 rests against the bottom of posterior rod 40, raising posterior rod 40 to the uppermost limit allowed by plunger rod guide 72. Anterior rod 42 is lowered to the lowermost limit allowed by plunger rod guide 72 due to tension from rubber band 24. Posterior rod 40 rests in long notch 46 against anterior ridge 48 to maintain plunger 22 in a posterior position. In a posterior position, plunger 22 extends out of tunnel 70 and holds rear frame 2 in a posterior position. Accordingly, engagement fixture 14 and pin 38 are also held in posterior positions. In this configuration, magnet 16 pulls friction lever 26 into an upright position. Friction lever 26 in an upright position aligns trail slot 53 perpendicular to friction trail 30.

Accordingly, friction lever 26 does not impede the movement of friction trail 30 in this configuration. For instance, friction trail 30 is free to move in an anterior direction, such as when lower frame 54 pivots in flexion, and friction trail 30 is free to move in a posterior direction, such as when lower frame 54 pivots in extension. Now with reference to FIG. 5B, when lower frame 54 pivots to its limit in flexion, the posterior arm of lower frame 54 pushes against rear frame 2. Accordingly, engagement fixture 14, pin 38 and plunger 22 are pushed as well. Plunger 22 is depressed into tunnel 70 and friction-based impedance module 10 enters a second configuration.

In a second state that starts in the swing phase when the knee maximally flexes and ends at the beginning of the stance phase when the knee starts flexing, friction-based impedance module 10 is primed and is ready to lock in response to a torque, such as from a rotation or weight load. At the start of this second state, and still with reference to FIG. 5B, the proximal edge of friction lever 26 rests against the bottom of posterior rod 40, raising posterior rod 40 to the uppermost limit allowed by plunger rod guide 72. Anterior rod 42 is lowered to the lowermost limit allowed by plunger rod guide 72 due to tension from rubber band 24. Posterior rod 40 rests in short notch 44 against posterior ridge 47 to maintain plunger 22 in an anterior position. In an anterior position, plunger 22 extends out of tunnel 70 and holds rear frame 2 in an anterior position. Accordingly, engagement fixture 14 and pin 38 are also held in anterior positions. Pin 38 in an anterior position holds friction lever 26 in an angled position, where the distal edge is anterior to the proximal edge. Friction lever 26 in an angled position also angles trail slot 53.

In this second state, the angled position of trail slot 53 allows friction trail 30 to move unimpeded in a posterior direction, such as when lower frame 54 pivots in extension. Now with reference to FIG. 5C, if friction trail 30 moves in an anterior direction, such as when lower frame 54 pivots in flexion, trail slot 53 catches friction trail 30 and friction-based impedance module 10 enters a third configuration.

In a third state that starts at the beginning of the stance phase and ends at the end of the weight acceptance phase around ˜40% through the gait cycle when the device is unloaded, friction-based impedance module 10 has locked in response to a weight load. As shown in FIG. 5D, the wearer's weight bearing upon the primed friction-based impedance module 10 applies a torque to cause lower frame 54 to pivot in flexion, moving friction trail 30 in an anterior direction. Trail slot 53 catches friction trail 30 and causes the proximal edge of friction lever 26 to undergo an anterior shift. The proximal edge of friction lever 26 pushes anterior rod 42 from underneath to the uppermost limit allowed by plunger rod guide 72. Posterior rod 40 falls by gravity to the lowermost limit allowed by plunger rod guide 72. Anterior rod 42 rests in long notch 46 against anterior ridge 48 to maintain plunger 22 in a medial position. In a medial position, plunger 22 extends slightly out of tunnel 70 and holds rear frame 2 in a medial position. Accordingly, engagement fixture 14 and pin 38 are also held in medial positions.

In this third state, as long as the wearer's weight remains upon friction-based impedance module 10 and maintains the torque, flexion by lower frame 54 will be locked. Removing the weight load removes the torque and shifts friction-based impedance module 10 back into the first configuration. Without the torque provided by the weight load, anterior rod 42 is pulled distally by the at least one rubber band 24. Posterior rod 40 is now pushed proximally by the proximal edge of friction lever 26. Plunger 22, having been released by the distal motion of anterior rod 42, is now pushed posteriorly by plunger spring 50. Plunger 22 stops posterior movement once posterior rod 40 seats in long notch 46 and rests against anterior ridge 48. Friction-based impedance module 10 has returned to the first configuration.

In some embodiments, the friction-based impedance module of the present invention further comprises a delay mechanism for delayed unlocking. For example, the mechanism may delay the friction-based impedance module from shifting from the third configuration to the first configuration upon the decrease or removal of a torque, such that friction-based impedance module remains locked for a brief period of time. A delay mechanism may be advantageous to enhance a user's ability to recover from a stumble in a lower extremity orthosis, or for tremor suppression in an upper extremity orthosis. In a moment of imbalance or weakness, the torque on a locked friction-based impedance module may be inadvertently removed. A delay mechanism allows the device to be able to provide a user with a brief period of time where the friction-based impedance module stays locked, giving the user the structural support needed to regain balance or suppress tremor.

In certain embodiments, the delay mechanism allows a limited range of motion in the friction-based impedance module upon the decrease or removal of a torque. For example, it may be advantageous for a user to have a functional range of motion for stumble recovery. The delay mechanism may restrict the friction-based impedance module to a safe range of motion for a brief period of time following the decrease or removal of a torque.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A friction-based impedance module, comprising:
an engagement fixture, a locking mechanism, a chassis and a lower frame;
wherein the chassis is connected to the lower frame by a pivoting joint;
wherein the module is configured to lock the pivoting joint responsive to an increased torque load, and wherein the module is configured to subsequently unlock responsive to a decreased torque load.

2. The friction-based impedance module of claim 1, wherein the locking mechanism is at least partially housed within the engagement fixture.

3. The friction-based impedance module of claim 1, wherein the locking mechanism comprises a plunger, a friction trail and a friction lever with a trail slot.

4. The friction-based impedance module of claim 3, wherein the friction trail passes through the trail slot of the friction lever.

5. The friction-based impedance module of claim 4, wherein the alignment of the trail slot with respect to the friction trail is controlled by the engagement fixture.

6. The friction-based impedance module of claim 5, wherein the trail slot contacts the friction trail to lock the pivoting joint.

7. The friction-based impedance module of claim 6, wherein the friction trail has a protrusion or void at an angle that prompts the engagement fixture into an engaged configuration.

8. The friction-based impedance module of claim 7, wherein the friction lever is released from the engaged configuration by a magnet, a spring, a solenoid, gravity, a motor, or movement of a user's limb.

9. The friction-based impedance module of claim 1, further comprising a mechanism for delaying the unlocking of the friction-based impedance module in response to a decreased torque load.

10. The friction-based impedance module of claim 1, wherein the locking mechanism is selected from the group consisting of: a ratchet and pawl mechanism, a roller clutch mechanism, a hydraulic mechanism, and a wrap spring clutch mechanism.

11. A hip-knee-ankle-foot orthosis (HKAFO), hip-knee-orthosis (HKO), knee-ankle-foot orthosis (KAFO), ankle foot orthosis (AFO), hip orthosis (HO), shoulder orthosis (SO), elbow orthosis (EO), wrist orthosis (WO), or knee orthosis (KO) comprising the friction-based impedance module of claim 1.

12. The friction-based impedance module of claim 1, further comprising at least one of a safety lock, configuration lock, dials, adjustment knobs, range of motion limits, dampers, and lubricating system.

13. The friction-based impedance module of claim 8, wherein the spring is replaced by a rubber band or other elastic material.

14. A serpentine spring device, comprising a plate having at least one S-shaped region.

15. The device of claim 14, wherein the device is composed of at least one material selected from the group consisting of: carbon fiber, a metal, and a composite material.

16. The device of claim 14, further comprising at least one additional stacked plate to modulate the flexibility of the device.

17. An orthotic device, comprising:
an impedance module including a chassis, a lower frame and a walking member;
wherein the chassis is coupled to the lower frame by a pivoting joint; and
wherein the walking member is coupled to the lower frame by a serpentine spring.

18. The orthotic device of claim 17, wherein the serpentine spring comprises at least one of a carbon fiber, metal, or composite material.

19. The orthotic device of claim 17, wherein the impedance module further comprises an engagement fixture and a locking mechanism, such that the module is configured to lock the pivoting joint responsive to an increased torque load, and wherein the module is configured to subsequently unlock responsive to a decreased torque load.

20. The orthotic device of claim 19, wherein the locking mechanism is at least partially housed within the engagement fixture.

21. The orthotic device of claim 19, wherein the locking mechanism comprises a plunger, a friction trail and a friction lever with a trail slot.

22. The orthotic device of claim 21, wherein the friction trail passes through the trail slot of the friction lever.

23. The orthotic device of claim 22, wherein the alignment of the trail slot with respect to the friction trail is controlled by the engagement fixture.

24. The orthotic device of claim 23, wherein the trail slot contacts the friction trail to lock the pivoting joint.

25. The orthotic device of claim 24, wherein the friction lever is released by a magnet, a spring, a solenoid, gravity, a motor, or movement of a user's limbs.

26. The orthotic device of claim 17, wherein the locking mechanism is selected from the group consisting of: a ratchet and pawl mechanism, a roller clutch mechanism, a hydraulic mechanism, and a wrap spring clutch mechanism.
